(19) United States
(12) Patent Application Publication
(10) Pub. No.: US 2008/0267935 A1
(43) Pub. Date: Oct. 30, 2008

(54) AraC in combination with a cytokine-secreting cell and methods of use thereof

(75) Inventors: JianMin Lin, Foster City, CA (US); Betty Li, San Francisco, CA (US); Eric Rimmer, Pacifica, CA (US); Melinda Van Roey, Oakland, CA (US); Karin Jooss, Bellevue, WA (US)

Correspondence Address: ROPES & GRAY LLP, PATENT DOCKETING 39/361, 1211 AVENUE OF THE AMERICAS, NEW YORK, NY, 10036-8704, US

(73) Assignee: Cell Genesys, Inc., South San Francisco, CA (US)

(21) Appl. No.: 12/080,719

(22) Filed: Apr. 4, 2008

Related U.S. Application Data
(60) Provisional application No. 60/922,102, filed on Apr. 6, 2007.

(51) Int. Cl. A61K 35/12; A61K 31/7068; A61P 35/04

(52) U.S. Cl. 424/93.71; 514/49

(57) ABSTRACT

The present invention provides improved method of cancer therapy in a mammal. More particularly, the invention is concerned with systems comprising cytosine arabinoside (AraC) and a cytokine-expressing cancer immunotherapy composition and methods of administering the combination to cancer patients in order to generate an immune response against the cancer and provide treatment with therapeutic efficacy that is an improvement relative to administration of AraC or the cytokine-expressing cancer immunotherapy composition alone as a monotherapy.

### CROSS-REFERENCE TO RELATED APPLICATIONS

This application claims benefit from Provisional Application No. 60/922,102 filed Apr. 6, 2007, the disclosures of which are hereby incorporated herein.

### FIELD OF THE INVENTION

The present invention relates to compositions and methods of preventing and/or treating cancer in a mammal. More particularly, the invention is directed to compositions and methods comprising a combination of cytosine arabinoside (AraC) and a cytokine-secreting cell and methods of administering the combination in order to treat cancer and generate a specific, long term immune response to cancer cells in a patient.

### BACKGROUND OF THE INVENTION

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

The use of cancer cells as vaccines to augment anti-cancer immunity has been explored for some time (Oettgen et al., “The History of Cancer Immunotherapy,” In: Biologic Therapy of Cancer, Devita et al. (eds.) J. Lippincot Co., pp. 87-199, 1991). However, due to the weak immunogenicity of many cancer cells, e.g., down regulation of MHC molecules, the lack of adequate costimulatory molecule expression and secretion of immunoinhibitory cytokines by cancer cells, the response to such vaccines has not resulted in long term efficacy. See, e.g., Armstrong T D and Jaffee E M, Surg Oncol Clin N Am. 11(3):681-96, 2002 and Bodey B et al., Anticancer Res 20(4):2665-76, 2000.

Numerous cytokines have been shown to play a role in regulating the immune response to tumors. For example, U.S. Pat. No. 5,098,702 describes using combinations of Tumor Necrosis Factor (TNF), Interleukin-2 (IL-2) and Interferon-β (IFN-β) in synergistically effective amounts to combat existing tumors. U.S. Pat. Nos. 5,078,996, 5,637,483 and 5,904,920 describe the use of Granulocyte macrophage colony-stimulating factor (GM-CSF) for treatment of tumors. However, direct administration of cytokines for cancer therapy may not be practical, as they are often toxic when administered systemically. (See, for example, Asher et al., J. Immunol. 146:3227-3234, 1991 and Havell et al., J. Exp. Med. 167:1067-1085, 1988.)

An expansion of this approach involves the use of genetically modified tumor cells which express cytokines locally at the vaccine site. Activity has been demonstrated in tumor models using a variety of immunomodulatory cytokines, including IL-4, IL-2, TNF-alpha, G-CSF, IL-7, IL-6 and GM-CSF, as described in Golumbeck P T et al., Science 254:13-716, 1991; Gansbacher B et al., J. Exp. Med. 172:1217-1224, 1990; Fearon E R et al., Cell 60:397-403, 1990; Gansbacher B et al., Cancer Res. 50:7820-25, 1990; Teng M et al., PNAS 88:3535-3539, 1991; Columbo M P et al., J. Exp. Med. 174:1291-1298,1991; Aoki et al., Proc Natl Acad Sci USA. 89(9):38504, 1992; Porgador A, et al., Nat Immun. 13(2-3): 113-30, 1994; Dranoff G et al., PNAS 90:3539-3543, 1993; Lee C T et al., Human Gene Therapy 8:187-193, 1997; Nagai E et al., Cancer Immunol, Immonther. 47:2-80, 1998 and Chang A et al., Human Gene Therapy 11:839-850,2000, respectively.

Clinical trials employing GM-CSF-expressing autologous or allogeneic cellular vaccines have commenced for treatment of prostate cancer, melanoma, lung cancer (e.g., non-small-cell lung carcinoma), pancreatic cancer, renal cancer, and multiple myeloma and have shown success (Dummer R., Curr Opin Investig Drugs 2(6):844-:8,2001; Simons J et al., Cancer Res. 15; 59(20):5160-8,1999; Soiffer R et al., PNAS 95:13141-13146,1998; Simons J et al., Cancer Res. 15; 57:1537-1546,1997; Jaffee E et al., J. Clin Oncol. 19:145-156,2001; and Salgia R et al., J. Clin Oncol. 21:624-630, 2003).

In yet another approach, autologous tumor cells were genetically altered to produce a costimulatory molecule, such as B7-1 or allogeneic histocompatibility antigens (Salvadori et al. Hum. Gene Ther. 6:1299-1306, 1995 and Plaksin et al. Int. J. Cancer 59:796-801, 1994).

Acute myeloid leukemias (AMLs) are highly malignant neoplasms responsible for a large number of cancer-related deaths. AML is a cancer of the myeloid line of white blood cells, characterized by the rapid proliferation of abnormal cells which accumulate in the bone marrow and interfere with the production of normal blood cells. The American Cancer Society estimates that 11,930 individuals in the U.S. will be diagnosed with AML annually. AML is quite resistant to currently available treatments, and approximately 76% of these patients will die of their disease (Deschler and Lubbert Cancer 2006; 107(9):2099-107; Jemal et al. Cancer statistics, 2006;56(2): 106-30). The 5-year survival rates range from 36% in patients younger than 45 years to only 1.3% in patients older than 75 years (Lowenberg et al., The New England journal of medicine 1999;341(14):1051-62; Kern and Estey Cancer 2006;107(1):116-24). Remission induction therapies using cytosine arabinoside (AraC), a nucleotide analogue (at a dosage of, for example, 100 to 200 mg/m²/day for 5 to 10 days) induce complete remissions in ˜75% of younger adults and −50% in patients who are older than 60 (Jabbour et al., Mayo Clinic proceedings 2006;81 (2):247-60; Kayaga et al Gene therapy 1999;6(8):1475-81). However, without intensive post-remission therapies, greater than 95% of all AML patients are destined to relapse (Abou-Jawde et al. Leukemia & lymphoma 2006;47[4]:689-95; Stone, Seminars in hematology 2002;39[3 Suppl 2]:4-10).

Post-remission therapy options include repeated cycles of high-dose AraC (e.g., 10 injections of 3000 mg/m² AraC) or high dose myelo-ablative chemotherapy combined with either autologous or allogeneic stem cell transplant. Whereas dose intensification of AraC during consolidation has been shown to be associated with lower relapse rates in younger patients, treatment options for patients over the age of 60 are limited. Most of these patients do not tolerate high dose AraC-based consolidation chemotherapy regimens well. The dose-limiting toxicities of AraC are severe neutropenia and lymphopenia, which are associated with higher mortality rates (Stone et al. The New England journal of medicine 1995;332(25): 1671-7). Therefore, the anti-leukemic benefit of increasing the dose of AraC is offset by increased treatment-related mortality. Another post-remission therapeutic option is hematopoietic stem cell transplantion, but this is a difficult procedure, and is also not a good option for older patients due to excessive treatment-related mortality.

Given the limitations of AraC treatment, and that the use of genetically modified cancer cells as anti-cancer vaccines has met with success in treatment of some forms of cancer, there remains a need for improved treatment regimens with greater potency/efficacy and less side effects than the therapies currently in use.

### SUMMARY OF THE INVENTION

The invention provides compositions and methods for the treatment of cancer in a mammal, typically a human, by administering a combination comprising a cytokine-expressing cellular vaccine and cytosine arabinoside (AraC).

In one aspect of the invention, the cytokine expressing cellular vaccine expresses Granulocyte macrophage colony-stimulating factor (GM-CSF).

In another aspect of the invention, the cytokine-expressing cellular vaccine is rendered proliferation-incompetent by irradiation.

In yet a further aspect of the invention, administration of the combination results in enhanced therapeutic efficacy relative to administration of the cytokine-expressing cellular vaccine or AraC alone.

In yet another aspect of the invention, the cytokine-expressing cellular vaccine is administered subcutaneously, intratumorally, or intradermally.

In yet another aspect of the invention, AraC is administered intravenously.

In another aspect of the invention, AraC may be administered prior to, at the same time as, or following the administration of the cytokine-expressing cellular vaccine component of the combination.

In yet another aspect of the invention, AraC is administered prior to the administration of the cytokine-expressing cellular vaccine.

The invention further provides a combination, wherein the combination comprises cells that are autologous, allogeneic, or bystander cells.

In another aspect of the invention, the autologous, allogeneic, or bystander cell is rendered proliferation-incompetent by irradiation.

The invention further provides compositions, methods and kits comprising cytokine-expressing cellular vaccines in combination with AraC for use according to the description provided herein.

### BRIEF DESCRIPTION OF THE DRAWINGS

FIGS. 1A and 1B are schematic depictions that show tracking of tumor burden in a mouse acute myeloid leukemia tumor model using a Xenogen IVIS (FIG. 1A); with progress of the tumor monitored by photon counts starting on Day 7 post challenge (FIG. 1B).

FIGS. 2A and 2B are graphic depictions of the absolute neutrophil (FIG. 2A); and absolute lymphocyte (FIG. 2B); count in peripheral blood collected on days 1, 2, 3, 4, 6, 8 and 11.

FIGS. 3A and 3B are depictions of the effect of treatment with AraC and inactivated GM-CSF-secreting tumor cell (C1498 GM) in a mouse acute myeloid leukemia tumor model (C1498.luc cells) with a Kaplan-Meier survival plot showing the results of treatment with: HBSS (squares), AraC (diamonds, C1498.GM (closed circles), and AraC/C1498.GM (closed triangles) in a mouse acute myeloid leukemia tumor model (FIG. 3A); with comparison of tumor development imaged with Xenogen IVIS between a subject treated with saline buffer control (HBSS) or with AraC/C1498.GM combination therapy (FIG. 3B).

FIG. 4 is a graphic depiction of the results of a study directed to evaluating the tumor-specific memory response following administration of the combination of AraC and GM-CSF-secreting tumor cell in the mouse acute myeloid leukemia tumor model. after receiving the combination therapy, animals that survived the initial C1498.luc tumor challenge was rechallenged with a second, larger dose of the C1498.luc cells. The results are presented as percent tumor-free mice versus days post rechallenge.

FIGS. 5A-5F are graphic depictions of the results of an evaluation of the effect of treatment with HBSS, C1498.GM and AraC plus C1498.GM, respectively in a mouse acute myeloid leukemia tumor model, indicating ⁵¹Cr release assay of splenocytes cocultured with inactivated C1498.GM using C1498 as target cells (FIGS. 5A-C). The percentage of purified splenocytes positive for CDI07a (FIG. 5D), CD44hi/CD62L1o (FIG. 5E), and NKG2D (FIG. 5F), in the CD8 subpopulation, as determined by flow cytometry, is also shown.

### DETAILED DESCRIPTION OF THE INVENTION

The present invention represents improved compositions for the treatment of cancer. The compositions comprise cytosine arabinoside (AraC) and a cytokine-secreting cellular vaccine. The invention includes methods of administering the combination in order to enhance the immune response to tumor cells in a patient.

The invention is not limited to the specific compositions and methodology described herein. It is also to be understood that the terminology used herein is for the purpose of describing particular embodiments only, and is not intended to limit the scope of the present invention.

### Definitions

The terms “immune response” as used herein refers to any alteration in a cell of the immune system or any alteration in the activity of a cell involved in the immune response. Such alteration includes an increase or decrease in the number of various cell types, an increase or decrease in the activity of these cells, or any other changes which can occur within the immune system. Cells involved in the immune response include, but are not limited to, T lymphocytes, B lymphocytes, natural killer (NK) cells, macrophages, eosinophils, mast cells, dendritic cells and neutrophils. In some cases, the immune response is stimulated or enhanced and in other cases the immune response is suppressed. Stimulation of the immune system may include memory responses and/or future protection against subsequent antigen challenge.

The terms “cytosine arabinoside,” “Cytarabine,” “AraC” and the like as used herein refer to a nucleotide analog used as a cancer chemotherapeutic agent. That is, a chemical agent that is toxic to, induces apoptosis in, or blocks cell division of a cancer cell. AraC is a standard treatment traditionally used in the treatment of cancer, e.g., radiation. AraC is typically administered in the form of a chemical entity, provided in a pharmaceutically acceptable excipient. In a further aspect, AraC is an agent or treatment, when administered to a patient in combination with a cytokine-expressing cellular vaccine results in an improved therapeutic outcome for the patient under treatment.

The term “cytokine” or “cytokines” as used herein refers to the general class of biological molecules which effect/affect cells of the immune system. The definition is meant to include, but is not limited to, those biological molecules that act locally or may circulate in the blood, and which, when used in the compositions or methods of the present invention serve to regulate or modulate an individual's immune response to cancer. Exemplary cytokines for use in practicing the invention include but are not limited to IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, ICAM, macrophage colony stimulating factor (M-CSF), granulocyte colony stimulating factor (G-CSF) and granulocyte macrophage colony stimulating factor (GM-CSF).

The term “cytokine-expressing cellular vaccine” as used herein refers to a composition comprising a population of cells that has been genetically modified to express a cytokine, e.g., GM-CSF, and that is administered to a patient as part of a cancer treatment regimen. The cells of such a “cytokine-expressing cellular vaccine” comprise a cytokine-encoding DNA sequence operably linked to expression and control elements such that the cytokine is expressed by the cells. The cells of the “cytokine-expressing cellular vaccine” are typically tumor cells and may be autologous or allogeneic to the patient undergoing treatment and or may be “bystander cells” that are mixed with tumor cells, typically taken from the patient.

The term “operably linked” as used herein relative to a recombinant DNA construct or vector means nucleotide components of the recombinant DNA construct or vector are directly linked to one another for operative control of a selected coding sequence. Generally, “operably linked” DNA sequences are contiguous, and, in the case of a secretory leader, contiguous and in reading frame, however, some sequences, e.g., enhancers do not have to be contiguous.

As used herein, the term “gene” or “coding sequence” means the nucleic acid sequence which is transcribed (DNA) and translated (mRNA) into a polypeptide in vitro or in vivo when operably linked to appropriate regulatory sequences. A “gene” typically comprises the coding sequence plus any non-coding sequences associated with the gene (e.g., regulatory sequences) and hence mayor may not include regions preceding and following the coding region, e.g., 5′ untranslated (5′UTR) or “leader” sequences and 3′ UTR or “trailer” sequences, as well as intervening sequences (introns) between individual coding segments (exons). In contrast, a “coding sequence” does not include non-coding DNA.

The terms “gene-modified” and “genetically-modified” are used herein with reference to a cell or population of cells wherein a nucleic acid chain has been introduced into the cell or population of cells. The nucleic acid sequence may be heterologous to the cell(s), or it may be an additional copy or improved version (e.g., mutated) of a nucleic acid sequence already present in the cell(s). The cell(s) may be genetically modified by physical or chemical methods or by the use of recombinant viruses. Chemical and physical, and viral methods can be utilized. Several recombinant viral vectors which find utility in effective delivery of genes into mammalian cells include, for example, retroviral vectors, adenovirus vectors, adenovirus-associated vectors (AAV), herpes virus vectors, pox virus vectors. Non-viral means of introduction include, for example, naked DNA delivered via liposomes, receptor-mediated delivery, calcium phosphate transfection, electroporation, particle bombardment (gene gun), or pressure-mediated delivery may also be employed to introduce a nucleic acid chain into a cell or population of cells to render them “gene-modified” or “genetically-modified”.

As used herein, the terms “tumor”, “neoplasm” and “cancer” refer to a cell that exhibits a loss of growth control and forms unusually large clones of cells. Tumor, neoplasmic, or cancer cells may also have lost contact inhibition and may be invasive and/or have the ability to metastasize.

The term “antigen from a tumor cell” and “tumor antigen” and “tumor cell antigen” may be used interchangeably herein and refer to any protein, carbohydrate or other component derived from or expressed by a tumor cell which is capable of eliciting an immune response. The definition is meant to include, but is not limited to, whole tumor cells that express all of the tumor-associated antigens, tumor cell fragments, plasma membranes taken from a tumor cell, proteins purified from the cell surface or membrane of a tumor cell, or unique carbohydrate moieties associated with the cell surface of a tumor cell. The definition also includes those antigens from the surface of the cell which require special treatment of the cells to access.

As described herein, a “tumor cell line” comprises cells that were initially derived from a tumor. Such cells typically are immortalised (i.e., genetically modified to exhibit indefinite growth in culture).

The term “systemic immune response” as used herein means an immune response which is not localized, but affects the individual as a whole.

The term “gene therapy” as used herein means the treatment or prevention of a disease or medical condition, including cancer, by means of ex vivo or in vivo delivery, through viral or non-viral vectors, of compositions containing a recombinant genetic material.

The terms “inactivated cells,” “non-dividing cells” and “non-replicating cells” may be used interchangeably herein and refer to cells that have been treated rendering them proliferation incompetent, e.g., by irradiation. Such treatment results in cells that are unable to undergo mitosis, but retain the capability to express proteins such as cytokines or other cancer therapeutic agents. Typically a minimum dose of about 3500 rads is sufficient, although doses up to about 30,000 rads are acceptable. Effective doses include, but are not limited to 5000 to 10000 rads. Numerous methods of inactivating cells, such as treatment with Mitomycin C, are known in the art. Any method of inactivation which renders cells incapable of cell division, but allows the cells to retain the ability to express proteins is included within the scope of the present invention.

As used herein “treatment” of an individual or a cell is any type of intervention used in an attempt to alter the natural course of the individual or cell. Treatment includes, but is not limited to, administration of e.g., a cytokine-expressing cellular vaccine, or a cytokine-expressing cellular vaccine and at least one additional cancer therapeutic agent or treatment, and may be performed either prophylactically or subsequent to diagnosis as part of a primary or follow-up therapeutic regimen. Treatment is any type of intervention that can result in improved therapeutic outcome, which can include, but is not limited to, induction of cancer remission, reduction of cancer relapse, reduction of cancer cells, reduction of cancer growth, prolongation of subject life, palliative effects, or induction of immune response to cancer cells.

The term “administering” as used herein refers to the physical introduction of a composition comprising a cytokine-expressing cellular vaccine, or a cytokine-expressing cellular vaccine and at least one additional cancer therapeutic agent or treatment to a patient with cancer. Any and all methods of introduction are contemplated according to the invention, the method is not dependent on any particular means of introduction and is not to be so construed. Means of introduction are well-known to those skilled in the art, examples of which are provided herein.

The term “co-administering” or “co-administered”, as used herein means a process whereby a cytokine-expressing cellular vaccine and at least one additional cancer therapeutic agent (e.g., AraC) or treatment to a patient with cancer, are administered to the same patient. The cytokine-expressing cellular vaccine and AraC are generally administered sequentially with AraC administered prior to the cytokine-expressing cellular vaccine. However, the cytokine-expressing cellular vaccine and AraC may be administered simultaneously or at essentially the same time. If administration takes place sequentially, the cytokine-expressing cellular vaccine is typically administered after AraC. The cytokine-expressing cellular vaccine and AraC may be included in a therapeutic regimen where an additional cancer therapeutic agent or treatment is also co-administered. The additional cancer therapeutic agent or treatment may be administered simultaneously, at essentially the same time or sequentially to one or both of the cytokine-expressing cellular vaccine and AraC. The cellular vaccine, AraC and the additional agent or treatment may be administered one or more times and the number of administrations of each component of the combination may be the same or different. In addition, the cytokine-expressing cellular vaccine and AraC need not be administered at the same site.

The term “therapeutically effective amount” or “therapeutically effective combination” as used herein refers to an amount or dose of a cytokine-expressing cellular vaccine together and the amount or dose of an additional agent or treatment that is sufficient generate an improved therapeutic outcome. The amount of cytokine-expressing cellular vaccine in a given therapeutically effective combination may be different for different individuals, different tumor types and will be dependent upon the one or more additional agents or treatments included in the combination. The “therapeutically effective amount” is determined using procedures routinely employed by those of skill in the art such that an “improved therapeutic outcome” results.

As used herein, the terms “improved therapeutic outcome” and “enhanced therapeutic efficacy” relative to cancer refers to a slowing or diminution of the growth of cancer cells or a solid tumor, increased immune response against the cancer cells, or a reduction in the total number of cancer cells or total tumor burden. An “improved therapeutic outcome” or “enhanced therapeutic efficacy” relative to the patient means there is an improvement in the condition of the patient according to any clinically acceptable criteria, including an increase in time to tumor progression, an increase in life expectancy, or an improvement in quality of life.

The terms “individual,” “subject” as referred to herein is a vertebrate, preferably a mammal, and typically refers to a human.

The terms “cancer therapeutic agent,” “additional cancer therapeutic agent or treatment” and the like as used herein refer to any molecule or treatment that stimulates an anti-cancer response when used alone or in combination with a cytokine-expressing cellular vaccine (e.g., GVAX®). In one aspect, the additional cancer therapeutic agent is expressed by a recombinant tumor cell and may be an immunomodulatory molecule, i.e., a second cytokine. In another aspect, the additional cancer therapeutic agent is administered in the form of a protein or other chemical entity, e.g., an antibody or standard chemotherapeutic agent provided in a pharmaceutically acceptable excipient. In yet another aspect, the cancer therapeutic agent is a standard treatment traditionally used in the treatment of cancer, e.g., radiation. In a further aspect, the additional cancer therapeutic agent is an agent or treatment, which is typically not considered in the treatment of cancer, but which when administered to a patient in combination with a cytokine-expressing cellular vaccine results in an improved therapeutic outcome for the patient under treatment.

### General Techniques

The practice of the present invention will employ, unless otherwise indicated, conventional techniques of molecular biology, microbiology, cell biology, biochemistry and immunology, which are within the knowledge of those of skill of the art. Such techniques are explained fully in the literature, such as, “Molecular Cloning: A Laboratory Manual,” second edition (Sambrook et al., 1989); “Current Protocols in Molecular Biology” (F. M. Ausubel et al., eds., 1987); “Animal Cell Culture” (R. I. Freshney, ed., 1987), each of which is hereby expressly incorporated herein by reference.

### Cancer Targets

The methods and compositions of the invention provide an improved therapeutic approach to the treatment of cancer by administration of a cytokine-expressing cellular vaccine and AraC alone or in combination with another treatment to a patient with cancer.

“Cancer”, “Tumor”, or “Neoplasm” as used herein includes cancer localized in tumors, as well as cancer not localized in tumors, such as, for instance, cancer cells that expand from a local tumor by invasion (i.e., metastasis). The invention finds utility in the treatment of any form of cancer, including, but not limited to, cancer of the bladder, breast, colon, kidney, liver, lung, ovary, cervix, pancreas, rectum, prostate, stomach, epidermis; a hematopoietic tumor of lymphoid or myeloid lineage; acute myeloid leukemia; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

### Introduction Of Cytokine And Cancer Therapeutic Agent Into Cells

In one aspect of the invention, a nucleic acid chain (i.e., a recombinant DNA construct or vector) encoding a cytokine operably linked to a promoter is introduced into a mammalian cell. Any and all methods for introduction of a recombinant DNA construct or vector into a cell, or population of cells, typically tumor cells, are contemplated according to the invention.

The “vector” may be a DNA molecule such as a plasmid, virus or other vehicle, which contains one or more heterologous or recombinant DNA sequences, e.g., a nucleic acid sequence encoding a cytokine under the control of a functional promoter and in some cases further including an enhancer that is capable of functioning as a vector, as understood by those of ordinary skill in the art. An appropriate viral vector includes, but is not limited to, a retrovirus, a lentivirus, an adenovirus (AV), an adeno-associated virus (AAV), a simian virus 40 (SV-40), a bovine papilloma virus, an Epstein-Barr virus, a herpes virus, a vaccinia virus, a Moloney murine leukemia virus, a Harvey murine sarcoma virus, a murine mammary tumor virus, and a Rous sarcoma virus. Non-viral vectors are also included within the scope of the invention.

Any suitable vector can be employed that is appropriate for introduction of a recombinant DNA construct into eukaryotic tumor cells, or more particularly animal tumor cells, such as mammalian, e.g., human, tumor cells. Preferably the vector is compatible with the tumor cell, e.g., is capable of facilitating expression of the coding sequence for a cytokine by the tumor cell, and is stably maintained or relatively stably maintained in the tumor cell. Desirably the vector comprises an origin of replication and the vector mayor may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern and Berg, J., 1982), mycophenolic acid (Mulligan and Berg, 1980), puromycin, zeomycin, hygromycin (Sugden et al., 1985) or G418.

In practicing the methods of the present invention, a vector comprising a nucleic acid sequence encoding a cytokine may be transferred to a cell in vitro, preferably a tumor cell, using any of a number of methods which include but are not limited to electroporation, membrane fusion with liposomes, Lipofectamine treatment, high velocity bombardment with DNA-coated microprojectiles, incubation with calcium phosphate DNA precipitate, DEAE-dextran mediated transfection, infection with modified viral nucleic acids, direct microinjection into single cells, etc. Procedures for the cloning and expression of modified forms of a native protein using recombinant DNA technology are generally known in the art, as described in Ausubel, et al., 1992 and Sambrook, et al., 1989, expressly incorporated by reference, herein.

Reference to a vector or other DNA sequences as “recombinant” merely acknowledges the operable linkage of DNA sequences which are not typically operably linked as isolated from or found in nature. A “promoter” is a DNA sequence that directs the binding of RNA polymerase and thereby promotes RNA synthesis. “Enhancers” are cis-acting elements that stimulate or inhibit transcription of adjacent genes. An enhancer that inhibits transcription also is termed a “silencer”. Enhancers can function (i.e., be operably linked to a coding sequence) in either orientation, over distances of up to several kilobase pairs (kb) from the coding sequence and from a position downstream of a transcribed region. Regulatory (expression/control) sequences are operatively linked to a nucleic acid coding sequence when the expression/control sequences regulate the transcription and, as appropriate, translation of the nucleic acid sequence. Thus expression/control sequences can include promoters, enhancers, transcription terminators, a start codon (i.e., ATG) in front of the coding sequined, splicing signal for introns and stop codons.

Recombinant vectors for the production of cellular vaccines of the invention provide all the proper transcription, translation and processing signals (e.g., splicing and polyadenylation signals) such that the coding sequence for the cytokine is appropriately transcribed and translated in the tumor cells into which the vector is introduced. The manipulation of such signals to ensure appropriate expression in host cells is within the skill of the ordinary skilled artisan. The coding sequence for the cytokine may be under control of (i.e., operably linked to) its own native promoter, or a non-native (i.e., heterologous) promoter, including a constitutive promoter, e.g., the cytomegalovirus (CMV) immediate early promoter/enhancer, the Rous sarcoma virus long terminal repeat (RSV-LTR) or the SV-40 promoter.

Alternately, a tissue-specific promoter (a promoter that is preferentially activated in a particular type of tissue and results in expression of a gene product in that tissue) can be used in the vector. Such promoters include but are not limited to a liver specific promoter (111 CR, et al., Blood Coagul Fibrinolysis 8 Suppl 2:S23-30, 1997) and the EF-1 alpha promoter (Kim D W et al. Gene. 91(2):217-23,1990, Guo Z S et al. Gene Ther. 3(9):802-10, 1996; U.S. Pat. Nos. 5,266,491 and 5,225,348, each of which expressly incorporated by reference herein). Inducible promoters also find utility in practicing the methods described herein, such as a promoter containing the tet responsive element (TRE) in the tet-on or tet-off system as described (ClonTech and BASF), the metallothienein promoter which can be upregulated by addition of certain metal salts and rapamycin inducible promoters (Rivera et al., 1996, Nature Med, 2(9):1028-1032; Ye et al., 2000, Science 283:88-91; Sawyer T K et al., 2002, Mini Rev Med Chem. 2(5):47588). Large numbers of suitable tissue-specific or regulatable vectors and promoters for use in practicing the current invention are known to those of skill in the art and many are commercially available.

Exemplary vector systems for use in practicing the invention include the retroviral MFG vector, described in U.S. Pat. No.5,637,483, expressly incorporated by reference herein. Other useful retroviral vectors include pLJ, pEm and [alpha]SGC, described in U.S. Pat. No. 5,637,483 (in particular Example 12), U.S. Pat. Nos. 6,506,604, 5,955,331-and U.S. Ser. No. 09/612808, each of which is expressly incorporated by reference herein.

Further exemplary vector systems for use in practicing the invention include second, third and fourth generation lentiviral vectors, U.S. Pat. Nos. 6,428,953, 5,665,577 and 5,981,276 and WO 00/72686, each of which is expressly incorporated by reference herein.

Additional exemplary vector systems for use in practicing the present invention include adenoviral vectors, described for example in U.S. Pat. No. 5,872,005 and WO 00/72686, each of which is expressly incorporated by reference herein.

Yet another vector system that is preferred in practicing the methods described herein is a recombinant adeno-associated vector (rAAV) system, described for example in W098/46728, WO 00/72686, Samulski et al., Virol. 63:3822-3828 (1989) and U.S. Pat. Nos. 5,436,146, 5,753,500, 6,037,177, 6,040,183 and 6,093,570, each of which is expressly incorporated by reference herein.

### Cytokines

Cytokines and combinations of cytokines have been shown to play an important role in the stimulation of the immune system. The term “cytokine” is understood by those of skill in the art, as referring to any immunopotentiating protein (including a modified protein such as a glycoprotein) that enhances or modifies the immune response to a tumor present in the host. The cytokine typically enhances or modifies the immune response by activating or enhancing the activity of cells of the immune system and is not itself immunogenic to the host.

Exemplary cytokines for use in practicing the invention include but are not limited to interferons (e.g., IFN-alpha, IFN-beta, and IFN-gamma), interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, macrophage colony stimulating factor (MCSF), granulocyte colony stimulating factor (G-CSF) and granulocyte-macrophage colony stimulating factor (GM-CSF). The cytokine may be from any source, however, optimally the cytokine is of murine or human origin (a native human or murine cytokine) or is a sequence variant of such a cytokine, so long as the cytokine has a sequence with substantial homology to the human form of the cytokine and exhibits a similar activity on the immune system. It follows that cytokines with substantial homology to the human forms of IFN-alpha, IFN-beta, and IFN-gamma, IL-1 to IL-29, TNF-alpha, TNF-beta, EPO, MIP3a, ICAM, M-CSF, G-CSF and GM-CSF are useful in practicing the invention, so long as the homologous form exhibits the same or a similar effect on the immune system. Proteins that are substantially similar to any particular cytokine, but have relatively minor changes in protein sequence find use in the present invention. It is well known that small alterations in protein sequence may not disturb the functional activity of a protein molecule, and thus proteins can be made that function as cytokines in the present invention but differ slightly from current known or native sequences.

### Variant Sequences

Homologues and variants of native human or murine cytokines are included within the scope of the invention. As used herein, the term “sequence identity” means nucleic acid or amino acid sequence identity between two or more aligned sequences and is typically expressed as a percentage (“%”). The term “% homology” is used interchangeably herein with the term “% identity” or “% sequence identity” and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence typically has greater than 80% sequence identity over a length of the given sequence. Preferred levels of sequence identity include, but are not limited to, 80, 85, 88, 89, 90, 91, 92, 93, 94, 95, 96, 97, 98 or 99% or more sequence identity to a native cytokine amino acid or nucleic acid sequence, as described herein.

Exemplary computer programs that can be used to determine the degree of identity between two sequences include, but are not limited to, the suite of BLAST programs, e.g., BLASTN, BLASTX, TBLASTX, BLASTP and TBLASTN, all of which are publicly available on the Internet. See, also, Altschul, S. F. et al. Mol. Biol. 215:403410, 1990 and Altschul, S. F. et al. Nucleic Acids Res. 25:3389-3402, 1997, expressly incorporated by reference herein. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. In determining sequence identity, both BLASTN and BLASTX (i.e., version 2.2.5) are run using default parameters of an open gap penalty of 11.0, and an extended gap penalty of 1.0, and utilize the BLOSUM-62 matrix. [See, Altschul, et al., 1997, supra.] A preferred alignment of selected sequences in order to determine “% identity” between two or more sequences, is performed using for example, the CLUSTAL-W program in Mac Vector version 6.5, operated with default parameters, including an open gap penalty of 10.0, an extended gap penalty of 0.1, and a BLOSUM 30 similarity matrix.

A nucleotide sequence is considered to be “selectively hybridizable” to a reference nucleotide sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about TM-5° C. (5° below the Tm of the probe) “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe, while high stringency conditions are used to identify sequences having about 80% or more sequence identity with the probe. An example of high stringency conditions includes hybridization at about 42° C. in 50% formamide, 5×SSC, 5× Denhardt's solution, 0.5% SDS and 100 fig/ml denatured carrier DNA followed by washing two times in 2×SSC and 0.5% SDS at room temperature and two additional times in 0.1×SSC and 0.5% SDS at 42° C. Moderate and high stringency hybridization conditions are well known in the art. See, for example, Sambrook, et al., 1989, Chapters 9 and 11, and in Ausubel, F. M., et al., 1993, (expressly incorporated by reference herein).

### Additional Cancer Therapeutic Agent Or Treatment

As detailed herein, the present invention is directed to a method of improving an individual's immune response to cancer (e.g., a target cancer antigen or antigens) by co-administering a cytokine-expressing cellular vaccine (e.g., GM-CSF) and AraC for treatment of a patient with cancer.

The methods of the invention may comprise the administration of an additional cancer therapeutic agent other than AraC for use in practicing the invention. Examples include, but are not limited to, adhesion or accessory molecules, other biological response modifiers, chemotherapeutic agents, radiation treatment and combinations thereof.

Embodiments of the present invention include therapeutic regimens for treatment of cancer comprising administration of the combination of a cytokine-expressing cellular vaccine and AraC.

### Cellular Vaccine

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, GM-CSF producing tumor cells are able to induce an immune response against themselves, as well as their parental, non-transduced tumor cell types.

Autologous and allogeneic cancer cells that have been genetically modified to express a cytokine, e.g., GM-CSF, followed by administration (or in the case of autologous cells, re-administration) to a patient for the treatment of cancer are described in U.S. Pat. Nos. 5,637,483, 5,904,920 and 6,350,445, expressly incorporated by reference herein. A form of GM-CSF-expressing genetically modified cancer cells or a “cytokine-expressing cellular vaccine” for the treatment of pancreatic cancer is described in U.S. Pat. Nos. 6,033,674 and 5,985,290, expressly incorporated by reference herein. A universal immunomodulatory cytokine-expressing bystander cell line is described in U.S. Pat. No. 6,464,973, expressly incorporated by reference herein. Clinical trials employing GM-CSF-expressing autologous or allogeneic cellular vaccines have been undertaken for treatment of prostate cancer, melanoma, lung cancer, pancreatic cancer, renal cancer, and multiple myeloma, and a number of these trials are currently ongoing.

Combination Therapy: Cytokine-Expressing Cellular Vaccine with AraC

The present invention provides an improved method of cancer therapy, which includes slowing the growth of or eradicating pre-existing malignancies as well as stimulating an immune response to cancer in a mammalian, preferably a human patient. Desirably, the method effects a systemic immune response, i.e., a T-cell response and/or a B-cell response, to the cancer. The method comprises administering to the patient a cytokine-expressing cellular vaccine and AraC, and may include another treatment. The cellular vaccine comprises cells which express a cancer antigen or various cancer antigens, the cancer antigen/antigens can be one of the antigens of the cancer found in the patient under treatment. The cells of the vaccine are rendered proliferation incompetent, for example by irradiation. Upon treatment, the cancer is eradicated, or its growth slowed, or enters remission, and an immune response against the cancer is elicited or enhanced. In one approach, the cytokine-expressing cellular vaccine combination comprises a single population of cells that is modified to express a cytokine which is co-administered with at least AraC. In another approach, the vaccine comprises two or more populations of cells individually modified to express one component of the vaccine, which are co-administered with AraC. In yet another approach, the cytokine-expressing cellular vaccine combination comprises a population of cells that is modified to express a cytokine which is administered with at least AraC. All of the above approaches, could also include the co-administration of additional treatments or therapeutic agents.

In general, a cytokine-expressing cellular vaccine for use in practicing the invention comprises tumor cells selected from the group consisting of autologous tumor cells, allogeneic tumor cells (including cell-lines) and non-tumor cell lines (e.g., bystander cells).

In some embodiments, the cells of the cytokine-expressing cellular vaccine are cryo-preserved prior to administration. In one aspect of the invention, the cells of the cytokine-expressing cellular vaccine are administered to the same individual from whom they were originally derived (autologous). In another aspect of the invention, the cells of the cytokine-expressing cellular vaccine and the tumor are derived from different individuals (allogeneic or bystander). In a preferred approach, the tumor being treated is selected from the group consisting of cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

In one aspect of the invention, the cells of the cytokine-expressing cellular vaccine comprises gene-modified cells of one type for the expression of the cytokine which are administered together with AraC. By way of example, in one approach, the cytokine-expressing cellular vaccine is provided as an allogeneic or bystander cell line delivered to the patient by the intradermal or subcutaneous route while AraC is injected intravenously. In another approach, the cytokine (i.e., GM-CSF) is expressed by autologous cells.

In previous studies, a direct comparison of murine tumor cells transduced with various cytokines demonstrated that GM-CSF-secreting tumor cells induced the best overall anti-tumor protection. In one preferred embodiment, the cytokine expressed by the cytokine-expressing cellular vaccine of the invention is GM-CSF. The preferred coding sequence for GM-CSF is the genomic sequence described in Huebner K. et al., Science 230(4731): 1282-5,1985. Alternatively the cDNA form of GM-CSF finds utility in practicing the invention (Cantrell et al., Proc. Natl. Acad. Sci., 82, 6250-6254, 1985).

Prior to administration, the cells of a cytokine-expressing cellular vaccine of the invention are rendered proliferation incompetent. While a number of means of rendering cells proliferation incompetent are known, irradiation is the preferred method. Preferably, the cytokine-expressing cellular vaccine is irradiated at a dose of from about 50 to about 200 rads/min, even more preferably, from about 120 to about 140 rads/min prior to administration to the patient. Most importantly, the cells are irradiated with a total radiation dose sufficient to inhibit growth of substantially 100% of the cells, from further proliferation. Thus, desirably the cells are irradiated with a total dose of from about 10,000 to 20,000 rads, optimally, with about 15,000 rads.

### Autologous Cellular Vaccine

The use of autologous cytokine-expressing cells in a vaccine of the invention provides advantages since each patient's tumor expresses a unique set of tumor antigens that can differ from those found on histologically-similar, MHC-matched tumor cells from another patient. See, e.g., Kawakami et al., J. Immunol., 148,638-643 (1992); Darrow et al., J. Immunol., 142,3329-3335 (1989); and Horn et al., J. Immunother., 10, 153-164 (1991).

In one embodiment, the present invention comprises a method of treating cancer by carrying out the steps of: (a) obtaining tumor cells from a mammal, preferably a human, harboring a tumor; (b) modifying the tumor cells to render them capable of producing a cytokine or an increased level of a cytokine naturally produced by the cells; (c) rendering the modified tumor cells proliferation incompetent; and (d) re-administering the modified tumor cells to the mammal from which the tumor cells were obtained or to a mammal with the same MHC type as the mammal from which the tumor cells were obtained. The administered tumor cells are autologous or MHC-matched to the host. AraC is co-administered to the mammal, typically prior to readministering modified cytokine-expressing tumor cells to the host.

A cancer treatment method of the invention may rely on the administration of one or more additional cancer therapeutic agents or treatments in addition to AraC and modified, cytokine-expressing tumor cells. The one or more additional cancer therapeutic agents may be expressed by the same autologous tumor cells that express the cytokine or the one or more additional cancer therapeutic agents may be expressed by a different autologous tumor cell population or by a different autologous tumor cell population using the same or a different vector. Alternatively, the therapeutic regime comprises administration of cytokine-expressing cells, AraC and one or more additional cancer therapeutic treatments such as irradiation or administration of a chemotherapeutic agent.

### Allogeneic Cellular Vaccines

In one preferred aspect, the invention provides a method for treating cancer by carrying out the steps of: (a) obtaining a tumor cell line; (b) modifying the tumor cell line to render the cells capable of producing a cytokine or an increased level of a cytokine naturally produced by the cells; (c) rendering the modified tumor cell line proliferation incompetent; and (d) administering the modified tumor cell line to a mammalian host having at least one tumor that is the same type of tumor as that from which the tumor cell line was obtained or wherein the tumor cell line and host tumor express at least one common antigen. The administered tumor cell line is allogeneic and is not MHC-matched to the host. Such allogeneic lines provide the advantage that they can be prepared in advance, characterized, aliquoted in vials containing known numbers of cytokine-expressing cells and stored such that well characterize cells are available for administration to the patient. Methods for the production of gene-modified allogeneic cells are described for example in WO 00/72686A1, expressly incorporated by reference herein. AraC is typically administered to the mammal prior to administering the modified allogeneic cytokine-expressing tumor cells to the host.

In one approach to preparing a cytokine-expressing cellular vaccine comprising gene-modified allogeneic cells, a cytokine-encoding nucleic acid sequence is introduced into a cell line that is an allogeneic tumor cell line (i.e., derived from an individual other than the individual being treated). In another approach, a cytokine-encoding nucleic acid sequence and the coding sequence for one or more additional cancer therapeutic agents are introduced into separate (i.e., different) allogeneic tumor cell lines. The cell or population of cells may be from a tumor cell line of the same type as the tumor or cancer being treated. The tumor and/or tumor cell line may be from any form of cancer, including, but not limited to, carcinoma of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; or another tumor, including a melanoma, teratocarcinoma, neuroblastoma, glioma, adenocarcinoma and non-small lung cell carcinoma.

In one aspect of the invention, the allogeneic tumor cell is modified by introduction of a vector comprising a nucleic acid sequence encoding a cytokine, operably linked to a promoter and expression control sequences necessary for expression thereof. In another aspect, the same allogeneic tumor cell or a second allogeneic tumor cell is modified by introduction of a vector comprising a nucleic acid sequence encoding an additional cancer therapeutic agent or treatment operably linked to a promoter and expression control sequences necessary for expression thereof. The nucleic acid sequence encoding the cytokine and additional cancer therapeutic agent or treatment may be introduced into the same or a different allogeneic tumor cell using the same or a different vector. The nucleic acid sequence encoding the cytokine or cancer therapeutic agent or treatment mayor may not further comprise a selectable marker sequence operably linked to a promoter.

Desirably, the allogeneic cell line expresses GM-CSF in a range from 200-1000 ng/10⁶ cells/24 h. Preferably, the universal bystander cell line expresses at least about 200 ng GM-CSF/10⁶ cells/24 hours.

In one embodiment of the invention, one or more allogeneic cell lines are incubated with an autologous cancer antigen, e.g., an autologous tumor cell (which together comprise an allogeneic cell line composition), then the allogeneic cell line composition is administered to the patient. Typically, the cancer antigen is provided by (on) a cell of the cancer to be treated, i.e., an autologous cancer cell. In such cases, the composition is rendered proliferation-incompetent by irradiation, wherein the allogeneic cells and cancer cells are plated in a tissue culture plate and irradiated at room temperature using a Cs source, as detailed above. The ratio of allogeneic cells to autologous cancer cells in a given administration will vary dependent upon the combination.

Any suitable route of administration can be used to introduce an allogeneic cell line composition into the patient, preferably, the composition is administered subcutaneously or intratumorally.

The use of allogeneic cell lines in practicing present invention provides the therapeutic advantage that, through administration of a cytokine-expressing allogeneic cell line and at least AraC to a patient with cancer, in the presence of an autologous cancer antigen, paracrine production of an immunomodulatory cytokine results in an effective immune response to a tumor. This obviates the need to culture and transduce autologous tumor cells for each patient, eliminating the problem of variable and inefficient transduction efficiencies.

### Bystander Cells in Cellular Vaccines

In one further aspect, the present invention provides a therapeutic treatment regimen which includes administration of AraC in combination with a universal bystander cell line that has been transduced to express an immunomodulatory cytokine. In some cases, the universal bystander cell line may express both a cytokine and one or more additional cancer therapeutic agents or each may be expressed by a different universal bystander cell line. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect of the invention, a universal bystander cell line is modified by introduction of a vector comprising a nucleic acid sequence encoding a cytokine operably linked to a promoter and expression control sequences necessary for expression thereof. In another aspect, the same universal bystander cell line or a second universal bystander cell line is modified by introduction of a vector comprising a nucleic acid sequence encoding one or more additional cancer therapeutic agents operably linked to a promoter and expression control sequences necessary for expression thereof. The nucleic acid sequence encoding the cytokine and additional cancer therapeutic agent(s) may be introduced into the same or a different universal bystander cell line using the same or a different vector.

In some cases, the bystander approach is combined with the autologous or allogeneic approach. For example, an autologous, allogeneic or bystander cell line encoding a cytokine may be co-administered with AraC and an autologous, allogeneic or bystander cell line encoding one or more additional cancer therapeutic agents. The nucleic acid sequence encoding the cytokine or additional cancer therapeutic agent(s) may or may not further comprise a selectable marker sequence operably linked to a promoter. Any combination of a cytokine, AraC and one or more additional cancer therapeutic agents that stimulate an anti-tumor immune response finds utility in the practice of the present invention. The universal bystander cell line preferably grows in defined, i.e., serum-free, medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of human bystander cell lines is described for example in U.S. Pat. No. 6,464,973 and WO 99/38954. Desirably, the universal bystander cell line expresses the cytokine, e.g., GM-CSF in the range from 200-1000 ng/10⁶ cells/24 h. Preferably, the universal bystander cell line expresses at least about 200 ng GM-CSF/10⁶ cells/24 hours.

In one embodiment of the invention, a universal bystander cell line is incubated with a cancer antigen, e.g., an autologous tumor cell or an allegeneic tumor cell line, which together comprise a universal bystander cell line composition. This universal bystander cell line composition is then administered to the patient. Any suitable route of administration can be used to introduce a universal bystander cell line composition into the patient. Preferably, the composition is administered subcutaneously or intratumorally.

Typically, the cancer antigen is provided by (on) a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed herein.

The ratio of bystander cells to autologous or allogeneic cancer cells in a given administration will vary dependent upon the combination. With respect to GM-CSF-producing bystander cells, the ratio of bystander cells to autologous cancer cells in a given administration should be such that at least 36 ng GM-CSF/10⁶ cells/24 hrs is produced. In general, the therapeutic effect is decreased if the concentration of GM-CSF is less than this. In addition to the GM-CSF threshold, appropriate ratios of bystander cells to autologous tumor cells or tumor antigens can be determined using routine methods in the art. In one embodiment, the ratio of bystander cells to autologous cancer cells should not be greater than 1:1.

The use of allogenic cancer cells or bystander cell lines in practicing the present invention provides the therapeutic advantage that it obviates the need to culture and transduce autologous tumor cells for each patient, eliminating the potential problem of variable and inefficient transduction efficiencies.

### AraC

AraC (1-β-D-arabinofuranosylcytosine) is one of the older chemotherapy drugs. It is described in U.S. Pat. No. 3,116,282, issued Dec. 31, 1963. It is a clear, colorless liquid given by the intravenous, intrathecal, intraperitoneal or subcutaneous route.

AraC has a CA registry number of 147-94-4. Its CA name is 4-amino-1-beta-D-arabinofuranosyl-2(1H)-pyrimidinone or 1-beta-D-arabinofuranosylcytosine. AraC is also known as beta-cytosine arabinosid, aracytidine, Alexan, Arabitin, Aracytine, Cytarbel, Cytosar, or Udicil.

AraC is most commonly used in treatment of acute myeloid leukemia, chronic myeloid leukemia, acute lymphoid leukemia and lymphomas.

AraC is listed as an antineoplastic or antimetabolite, a class of drugs that interfere with DNA and RNA. AraC's anti-cancer activity is associated with its ability to be converted to its biologically active form, AraCTP. However, AraC is only slowly converted to AraCTP in the liver or in primary liver tumors due to low levels of an enzyme in the liver that is required for the conversion of AraC to AraCMP, the first step in the activation pathway of the drug. Higher doses of AraC cannot be used to overcome this limitation due to bone marrow toxicity resulting from rapid activation in that tissue.

The degree and severity of the side effects depend on the amount and schedule of Ara-C administration. Some of the most common side effects of AraC treatment include low white blood counts, low platelet count, anemia, hair loss, soreness of the mouth, difficulty swallowing, and diarrhea. In the treatment of AML, even after initial AraC treatments result in a complete remission of the cancer in the patient, the chance of cancer recurrence is high.

### Evaluation Of Combinations In Animal Models C1498-luc Tumor Model

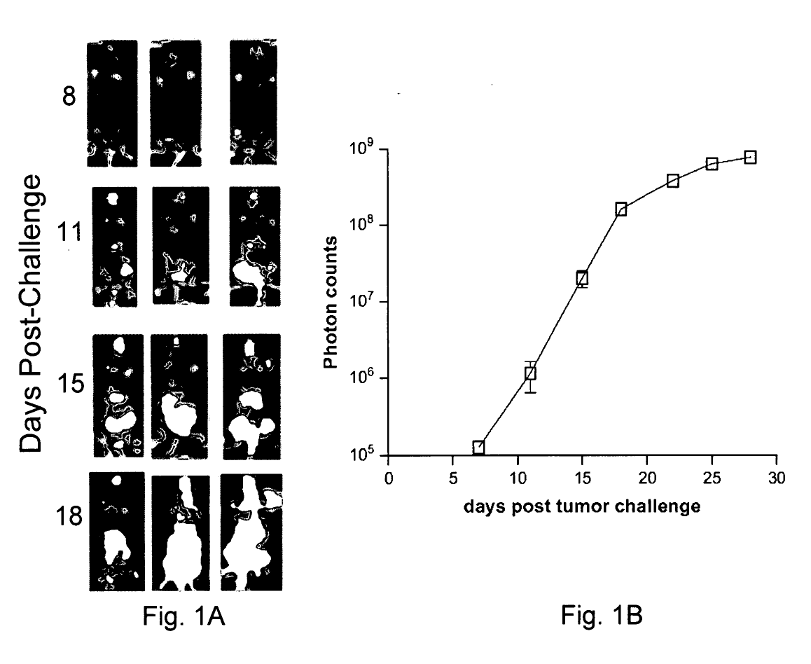

The C1498-luc tumor model was developed to evaluate the effects of a GM-CSF-secreting tumor cell vaccine, C1498.GM, in combination with AraC. C1498 is a murine AML tumor-derived cell line, and its administration is an often-used model of AML. In order to monitor leukemia progression in the animals, C1498 cells were first transduced with a lentiviral vector encoding the luciferase reporter gene to create the C1498-luc subline. To assess the in vivo progression of systemic disease, 2.5×10⁴ of C1498-luc cells were injected intravenously via tail vein into C57BL/6 mice, their syngeneic host, and the mice were examined every few days for the presence of luminescent signal via live imaging (FIG. 1A). C1498-luc tumor cells were visualized in the lungs minutes post injection. The tumor cells then dispersed from the lungs to lymph nodes and bones and the disease progressed aggressively to detectable systemic lesions within 15 days. In FIG. 1B, animals with photon counts post-tumor challenge are shown. Whole body photon counts per mouse increased from approximately 5×10⁴ one week post inoculation to greater than 5×10⁸ three weeks post inoculation. Animals with photon counts exceeding 5×10⁸ exhibited clinical symptoms including ascites, weight loss and paralysis. Necropsy data, from these animals showed tumor growth in bone marrow, lymph nodes, spleen, ovaries and ascites and luciferase-positive tumors were readily visualized by imaging the isolated organs (data not shown). A total photon count of 5×10⁸ from an individual tumor bearing animal was used as the end point of life expectancy. The survival of untreated mice was approximately three to four weeks with an MST of 27 days (data not shown).

The efficacy of the combination of cytosine arabinoside (AraC) and GM-CSF secreting cells was evaluated by carrying out animal studies in the syngeneic C1498-luc tumor model. In this model, following challenge with C1498-luc tumor cells as described above, the mice were randomized into control and individual treatment groups, as detailed in the examples. For anti-tumor memory assessment, animals which had previously received AraC in combination with GM-CSF-secreting cells were rechallenged with a lethal dose of C1498-luc cells approximately 100 days after receiving the combination therapy. Tumor progression was monitored by Xenogen imaging in vivo, as shown in FIG. 1A. A typical study in the C1498-luc tumor model makes use of at least 6 and generally 10-15 mice per group in order to obtain statistically significant results. Statistical significance is evaluated using the Student's t-test.

Immunotherapy with inactivated tumor cells engineered to secrete GM-CSF is known to elicit long-term systemic, tumor-specific immune responses. For example, after mice were injected with inactivated B16 cells (C57BL/6 mouse melanoma cell line) virally transduced to express GM-CSF, subsequent injections of wild-type, non-inactivated B16 cells did not result in tumor formation. In contrast, injection of wild type inactivated B16 cells (not expressing GM-CSF) did not protect the mice from subsequent introduction of live B16 cells, showing the importance of GM-CSF expression in triggering the immune response.

Vaccination of C57BL/6 mice with irradiated GM-CSF-secreting C1498 tumor cells stimulated potent, long-lasting and specific anti-tumor immunity that prevented tumor growth in most mice subsequently challenged with wild-type C1498 cells. Previous experiments have demonstrated that HBSS or irradiated B16F10 alone do not protect challenged mice from tumor formation. In addition, although mice treated with GM-CSF-expressing B16 cells were protected from subsequent challange with wild-type non-inactivated B16, they were not protected from challenge of Lewis Lung carcinoma cells (another tumor of C57BL/6 origin). Similarly, GM-CSF-expressing Lewish Lung carcinoma cells did not protect mice from a challence of live, wild type B16 cells.

The combination of a cytokine-expressing cellular vaccine plus AraC treatment is expected to increase the efficacy of tumor treatment and subsequent protection. However, the degree of increased efficacy that could be expected was not clear, as the efficacy depends on several factors such as the doses of AraC and the cytokine-expressing cellular vaccine, as well as the inclusion of another treatment (i.e., dose of the agent or the frequency and strength of radiation) in the therapeutic treatment regime. The relative timing and route of administration of relative to the timing of administration of the cytokine-expressing cellular vaccine could also impact the therapeutic outcome. Another concern was that the lymphopenia and neutropenia caused by AraC could potentially interfere with the development of the long-term immune response through the vaccination.

### Immunological Monitoring

Several tumor associated antigens have been identified which allow one to monitor tumor as well as antigen specific immune responses. For example, tumor antigen-specific T cells can be identified by the release of IFN-gamma following antigenic restimulation in vitro (Hu, H-M. et al., Cancer Research, 2002, 62; 3914-3919). Yet another example of new methods used to identify tumor antigen-specific T cells is the development of soluble MHC I molecules also known as MHC tetramers (Beckman Coulter, Immunomics), reported to be loaded with specific peptides shown to be involved in an anti-tumor immune response. Examples within the C1498 model include, but are not limited to, gp100, Trp2, Trp-1, and tyrosinase. Similar melanoma-associated antigens have been identified in humans. Such tools provide information that can then be translated into the clinical arena.

### Assays For Efficacy Of Combination Therapy In Vivo Models

Tumor burden is assessed at various time points after tumor challenge. Typically, spleens cells are assessed for CTL activity by in vitro whole cell stimulation for 5 days. Target cells are labeled with ⁵¹Cr and co-incubated with splenic effector CTL and release of ⁵¹Cr into the supernatants as an indicator of CTL lysis of target cells. On day 3 of in vitro stimulated CTL supernatants are tested for IFN-gamma production by CTL. In brief, wells are coated with coating antibody specific for IFN-gamma, supernatant is then added to wells, and IFN-gamma is detected using an IFN-gamma specific detecting antibody. IFN-gamma can also be detected by flow cytometry, in order to measure cell-specific IFN-gamma production.

Another indication of an effective anti-tumor immune response is the production of effector cytokines such as TNF-alpha, IL-2, and IFN-gamma upon restimulation in vitro. Cytokine levels were measured in supernatants from spleen cells or draining lymph node (dLN) cells restimulated in vitro for 48 hours with irradiated GM-CSF-expressing cells.

A further method used to monitor tumor-specific T cell responses is via intracellular cytokine staining (ICS). ICS can be used to monitor tumor-specific T-cell responses and to identify very low frequencies of antigen-specific T-cells. Because ICS is performed on freshly isolated lymphocytes within 5 hours of removal, unlike the CTL and cytokine release assays, which often require 2-7 days of in vitro stimulation, it can be used to estimate the frequency of tumor antigen-specific T-cells in vivo. This provides a powerful technique to compare the potency of different tumor vaccine strategies. ICS has been used to monitor T-cell responses to melanoma-associated antigens such as gp1OO and Trp2 following various melanoma vaccine strategies. Such T-cells can be identified by the induction of intracellular IFN-gamma expression following stimulation with a tumor-specific peptide bound to MHC I.

### Xenogen Imaging of Tumor Models

In some studies, the development and spread of tumors is monitored by employing the Xenogen whole-animal imaging system. A cancer cell that has been transduced to express a fluorescent protein, such as luciferase, is transplanted into a subject, then cancer progression is monitored by recording in vivo luminescence of the tumor bearing mice. In brief, Balb/c nu/nu mice are injected with 5×10⁴ or 2×10⁵ cells of C1498-luc cells via tail vein on day O. Mice are monitored for tumor burden when necessary by intra-peritoneal injection of excess luciferin substrate at 1.5 mg/g mice weight. In a typical analysis, twenty minutes after substrate injection, mice are anesthesized and monitored for in vivo luminescence with Xenogen IVIS Imaging System (Xenogen Inc.) luminescence sensitive CCD camera by dorsal or ventral position. Data is collected and analyzed by Living Image 2.11 software.

### Cytokine-Expressing Cellular Vaccine Combinations

The present invention is directed to administration of the combination of a cytokine-expressing cellular vaccine and AraC to a cancer patient. The combination may be co-administered with an additional cancer therapeutic agent or treatment. The additional cancer therapeutic agent or treatment may be a chemotherapeutic agent, an agent that modulates the immune response to a cancer antigen, radiation, etc.

Co-Stimulatory Molecules in Combination with Cytokine-Expressing Cellular Vaccines

In natural immune responses, CD4+ T helper (Tₕ) cells, reactive with peptide antigens presented by MHC class II molecules on dendritic cells (DC), can drive the maturation of DC which is required for induction of CD8+ CTL immunity. Proper induction, expansion and maintenance of CTL responses are achieved through the interaction between CD4+ T cells, DC and CD8+ T cells. While the mechanism is not part of the invention, the cells to a large extent operate through up-regulation of CD40L, which interacts with DC-expressed CD40 to effect DC maturation. CD80/CD86 expressed by mature or activated DC can effect CTL induction by interaction with the CS28 costimulatory receptor on CD8+ T cells. For maintenance and full expansion of CTL, interaction of the DC expressed 4-1BB ligand with its receptor 4-1BB on CTL is also important. DC activation may be triggered by e.g., agonistic anti-CD40 antibody or ligands of Toll-like receptors (TLR) such as LP5 (TLR4 ligand) or oligodeoxynucleotides containing CpG-motifs (TLR9 ligand).

### Cytokine-Expressing Cellular Vaccines Plus AraC

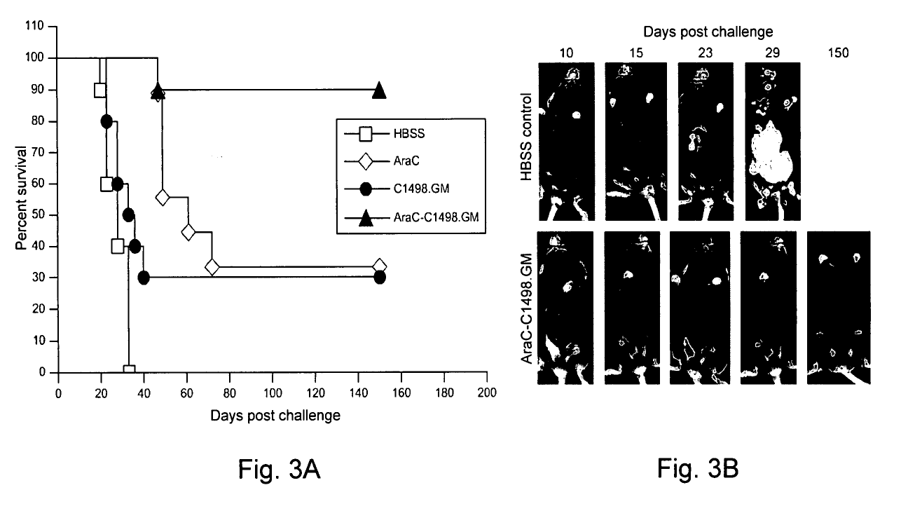

The results presented herein demonstrate that the combination of GM-CSF-secreting C57BL/6 tumor cells and AraC in the treatment of tumor-bearing subject act synergistically, resulting in significantly improved survival compared to either treatment being used as a monotherapy, as well as the establishment of long-term protective anti-tumor immune responses. In order to achieve the maximal synergistic effect of these two agents in clinical trials, it is essential to carefully evaluate possible treatment regimens in preclinical studies. In studies described herein, the efficacy of the combination was evaluated in preclinical studies following repeated administration of both AraC and GM-CSF-secreting tumor cell vaccines in vivo in a murine tumor model. Example 1 details hematological toxicity of AraC. Example 2 details studies where AraC and a cytokine-expressing cellular vaccine (GMCSF-secreting C57BL/6 tumor cells) were tested in the C57BL/6 model as monotherapies and as a combination therapy (FIG. 3). The combination therapy of AraC and a GM-CSF secreting tumor cell vaccine together was dramatically more effective in treating tumors than monotherapy regimens using AraC or the GM-CSF-secreting tumor cell vaccine separately(Table 1, FIGS. 3, 4, and 5A-5F).

These results demonstrate that in practicing the present invention an autologous, allogeneic, or bystander cytokine-expressing cellular vaccine may be administered to a cancer patient in combination with an AraC resulting in enhanced therapeutic efficacy and prolonged survival relative to either monotherapy alone.

In a preferred aspect of the methods described herein, a cytokine-expressing cellular vaccine combination is administered to a cancer patient, wherein the cytokine expressing cellular vaccine comprises mammalian, preferably human tumor cells, and the cells in the cytokine-expressing cellular vaccine are rendered proliferation incompetent, for example, by irradiation.

The cytokine-expressing cellular vaccine combination may be administered by any suitable route. Preferably, the composition is administered subcutaneously or intratumorally. Local or systemic delivery can be accomplished by administration comprising administration of the combination into body cavities, by parenteral introduction, comprising intramuscular, intravenous, intraportal, intrahepatic, peritoneal, subcutaneous, or intradermal administration. In the event that the tumor is in the central nervous system, the composition is administered in the periphery to prime naive T-cells in the draining lymph nodes. The activated tumor-specific T-cells are able to cross the blood/brain barrier to find their targets within the central nervous system.

In one exemplary embodiment, the cytokine-expressing cellular vaccine is GM-CSF-expressing cellular vaccine, where the cytokine expressed is GM-CSF.

As will be understood by those of skill in the art, the optimal treatment regimen will vary. As a result, it will be understood that the status of the cancer patient and the general health of the patient prior to, during, and following administration of a cytokine-expressing cellular vaccine combination, the patient will be evaluated in order to determine if the dose of each component and relative timing of administration should be optimized to enhance efficacy or additional cycles of administration are indicated. Such evaluation is typically carried out using tests employed by those of skill in the art to evaluate traditional cancer chemotherapy, as further described below in the section entitled “Monitoring Treatment.”

### Monitoring Treatment

One skilled in the art is aware of means to monitor the therapeutic outcome and/or the systemic immune response upon administering a combination treatment of the present invention. In particular, the therapeutic outcome can be assessed by monitoring attenuation of tumor growth and/or tumor regression and or the level of tumor specific markers. The attenuation of tumor growth or tumor regression in response to treatment can be monitored using several end-points known to those skilled in the art including, for instance, number of tumors, tumor mass or size, or reduction/prevention of metastasis.

All literature and patent references cited above are hereby expressly incorporated by reference herein.

### Materials and Methods

Cell Lines and Reagents. C1498, a murine AML cell line, was purchased from American Type Culture Collection (ATCC, Manassas, Va.). C1498 was originally derived from a female C57Bl/6J (H-2b) mouse and subsequently adapted to tissue culture and is MHC class I⁺ and MHC class II⁺. The C1498-luc subline was established by transduction of C1498 with lentiviral vector expressing a luciferase reporter gene, and the C1498.GM subline by transduction of C1498 with lentiviral vector expressing mouse GM-CSF. The latter generates 70 ng of mouse GM-CSF per 10⁶ cells per 24 hours in culture. Both transduced cell lines were maintained in culture conditions recommended by ATCC. Cytarabine, also known as cytosine arabinoside or AraC, was purchased from Cardinal Health, San Diego, Calif.

Mice. Female C57Bl/6 mice and female C57Bl/6 congenic Thy 1.1 mice were purchased from Taconic (Oxnard, Calif.) and the Jackson Laboratory (Bar Harbor, Me.) respectively, and maintained according to institutional and NIH guidelines. All mice were used between 8 and 12 weeks of age. Water and food were provided ad libitum.

Tumor Model. Female C57Bl/6 mice were challenged with C1498-luc cells via intra-tail-vein injections with 2.5×10⁴ inocula. The mice were prepared for in vivo bioluminescence imaging 5 to 10 minutes post injection to confirm the initial trafficking of the tumor cells from tail vein to the lungs. Briefly, the mice were injected i.p. with 1.5 mg/g luciferin substrate (Xenogen Corp., Alameda, Calif.). Fifteen minutes later, the mice were anesthetized for in vivo bioluminescence imaging analysis. Nearly 100% of challenged mice imaged positively, demonstrating initial trafficking of C1498-luc to the lungs. The animals were monitored by in vivo imaging every 5 to 7 days throughout the study to monitor the systemic progression of the tumor. Individual animals were euthanized when in vivo total photon counts exceeded 5×10⁸ and/or when determined to be clinically paralyzed.

Hematologic and Phenotypic Analysis. Mice were injected intraperitoneally with AraC using the treatment regimen described below. Peripheral blood was collected by retro-orbital puncture into EDTA-coated capillary tubes on days 1, 2, 3, 4, 6, 8 and 11. Hematologic analysis was performed by IDEXX pre-clinical Research Services (West Sacramento, Calif.).

In Vivo Treatment. Following challenge with C1498-luc tumor cells as described above, the mice were randomized into control and individual treatment groups. For AraC treatment; at 24 hours post challenge, the animals received three i.p. injections of 100 mg/kg AraC (volume of injection: 200 μl) in 10 hour-increments. This treatment regimen is equal to a total dose of 900 mg/m² (300 mg/m² per injection) and is within the total dose range of 700 to 1400 mg/m² used clinically in human patients. This dose level is typical for inducing remission, which is a dosage substantially lower than a typical post-remission high-dose consolidation regimen (e.g., 10 doses of 3000 mg/m² for a total dose of 30,000 mg/m²). For C1498.GM treatment, 7 days post challenge; the animals were given a single dose of irradiated C1498.GM cells at 1×10⁶/500 Jμl subcutaneously. For the combination therapies, 3 AraC injections at 10-hour intervals were given on day 2 followed by a single C1498.GM injection 3, 5, or 7 days post AraC, at the nadir, rebound or at the recovered phase of lymphopenia and neutropenia induced by AraC, respectively. For long-term anti-tumor memory assessment, animals which had previously received AraC in combination with GM-CSF-secreting Tumor Cell Immunotherapy were rechallenged with a lethal dose of 5×10⁴ C1498-luc cells approximately 100 days after receiving the combination therapy. A group of five naive mice were received the same inoculum of C1498.luc as control. Tumor progression was monitored by Xenogen imaging in vivo.

Flow Cytometric Analysis. Splenocytes from mice (n=5/group) were harvested and mechanically dissociated using glass slides. C1498 tumor cells were depleted using anti-thyl.2 MACs beads. Cells were counted and single cell populations of tumor-cell and erythrocyte-depleted spleen cells were stained with conjugated antibodies purchased from BD Pharmingen (San Diego, Calif.). 30,000 gated events were collected on a FACSCAN (Becton Dickinson) and analyzed using CellQuest software (Becton Dickinson).

⁵¹Cr Release Cytotoxicity Assay. Activity of cytotoxic T-Iymphocytes (CTL) was assessed using the standard ⁵¹Chromium-release assay. Briefly, 2×10⁶ target cells were labeled at 37° C. for 1 h with 100 μCi Na₂⁵¹CrO4 (MP Biomedicals). Target cells were washed 3× and resuspended to 5×10⁴ cells/ml. Five thousand radiolabeled target cells per well (100 μl) were added to a 96 well plate, together with the appropriate number of effector cells (100 μl/well). The defined effector:target (E:T) ratios were plated in triplicate. Cytotoxicity assays were performed at 37° C. for 4 h. After incubation, cell-free supernatants were collected and analyzed in a gamma counter. Percent specific lysis was calculated using the following equation: (ER−SR)/(MR−SR)×100, where ER=experimental release, SR=spontaneous release and MR=maximum release.

### EXAMPLE 1

### Characterizing Hematological Toxicity of AraC Treatment

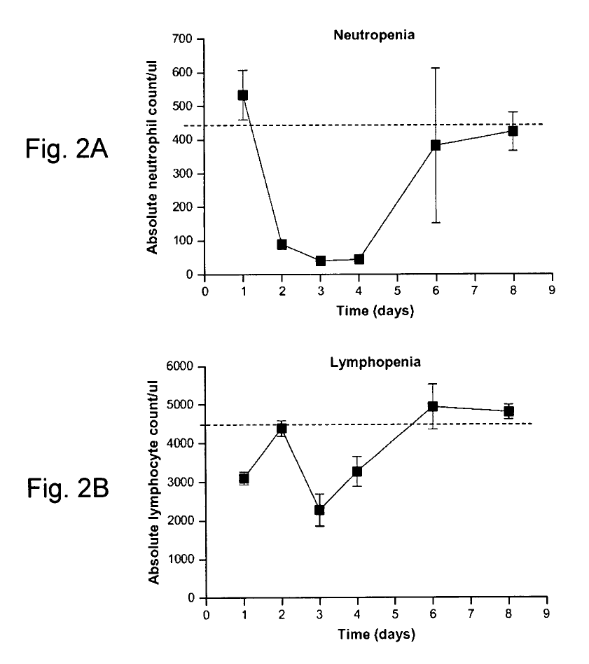

Prior to conducting the in vivo efficacy studies in the leukemia tumor models, the hematological toxicity of AraC was determined. The animals received three i.p. injections of 100 mg/kg AraC (volume of injection: 200 μl) in 10 hour-increments. This treatment regimen is equal to a total dose of 900 mg/m² and is within the total dose range of 700 to 1400 mg/m² used clinically in human patients. Neutropenia and lymphopenia are known to be the primary dose-limiting toxicity observed in patients, so AraC treated mice were monitored for absolute neutrophil and lymphocyte counts. The results are shown in FIG. 2A and FIG. 2B, respectively. After the three AraC administrations, peripheral blood was collected by retro-orbital puncture into EDTA-coated capillary tubes on days 1, 2, 3, 4, 6, 8 and 11 and analyzed for neutropenia. The absolute neutrophil count in the animals dropped to 45 neutrophils/μl, by day 4, rebounded to control level at 600 neutrophils/μl, by day 6 and remained at a steady level of around 600 neutrophils/μl, thereafter (FIG. 2A), which is similar to untreated control animals. Similar effects were observed on lymphocyte counts, which dropped by greater than 40% by day 3 and rebounded by day 6 (FIG. 2B).

### EXAMPLE 2

### Combination Therapy: Cytokine-Expressing Cellular Vaccine and AraC

In vivo studies were carried out using the C57BL/6 model to determine if AraC in combination with a cytokine-expressing cellular vaccine can enhance anti-cancer efficacy. The optimal timing of AraC administration relative to GM-CSF-expressing cellular vaccine was also investigated.

C57BL/6 mice were inoculated intravenously via tail vein on day 0 with 2.5×10⁴ C1498-luc cells expressing the luciferase reporter gene. The C1498-luc tumor bearing mice were treated with either AraC or C1498.GM or in combination. For monotherapy, AraC was administered one day post tumor challenge by three intraperitoneal injections at 100 mg/kg 10 hours apart or 1×10⁶ irradiated C1498.GM cells were administered as a single subcutaneous injection to designated animals on day 7 post tumor challenge. The animals receiving combination therapies were given the two agents sequentially, scheduled as for monotherapy. The dose levels of AraC and C1498.GM given in this experiment did not result in substantial anti-tumor activity in the C1498-luc tumor model when used as monotherapy, however, detection of synergistic effects of the two therapies were readily detectable (FIG. 3). At the dose levels used in this experiment, both AraC and C1498.GM as monotherapies had modest positive effects on survival of C1498-luc tumor bearing animals, with only 30% of animals surviving the disease at 150 days post-challenge. The combination of AraC and C1498.GM significantly prolonged survival of tumor bearing mice over either monotherapy regimens. The majority (90%) of mice receiving the combined therapy survived for longer than 150 days, while only 30% of mice receiving either AraC or c1498.GM monotherapy survived for over 150 days. In addition, the surviving mice of the combination therapy group were tumor free at the 150 day time point, while none of the monotherapy mice were, as shown in Table 1.

TABLE 1
Median Survival Time (MST) of Mice Treated With
AraC or C1498.GM Alone or In Combination.

| | Time of C1498.GM | | Number of |
|---|---|---|---|
| | administration | | tumor free |
| | (days post AraC | MST | animals 150 days |
| Treatment | treatment) | (days) | post challenge |
| HBSS | — | 28 | 0/10 |
| AraC | — | 61 | 0/10 |
| C1498.GM | N/A | 35 | 0/10 |
| AraC + C1498.GM | 3 | >150 | 9/10 |
| AraC + C1498.GM | 5 | >150 | 10/10 |
| AraC + C1498.GM | 7 | >150 | 10/10 |

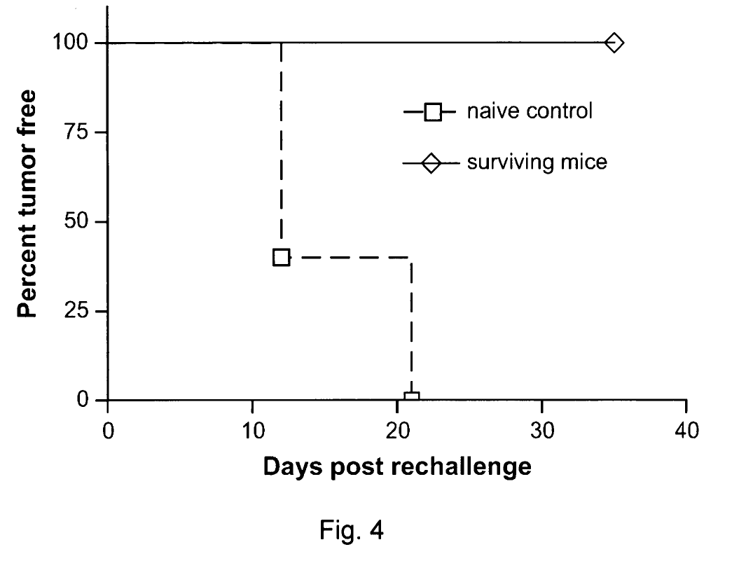

Mice surviving from the combination therapy group (n=10) then underwent tumor challenge again, with 5×10⁴ live C1498-luc cells, a lethal dose, administered on day 143 after receiving the combination therapy. Upon rechallenge, none of the animals that previously received combination therapy developed tumors, and they remained tumor-free for the duration of the study (>100 days), demonstrating the existence of a long term protective response against the cancer cells (FIG. 4). The presence of this long-term protection would also prevent a post-remission recurrence of the cancer.

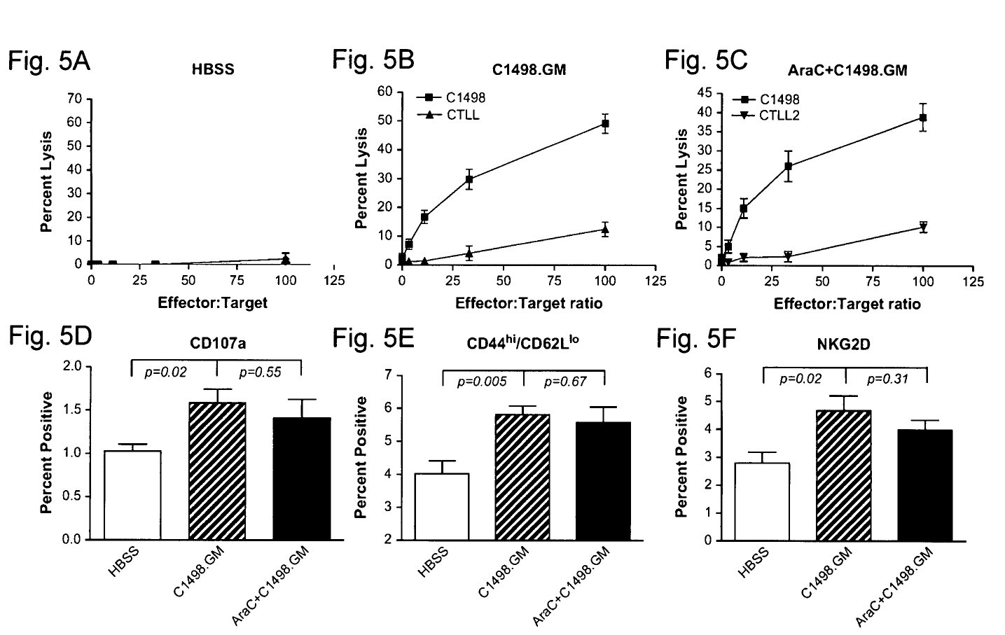

Tumor-specificity of T-cell responses were evaluated in animals treated with C1498.GM monotherapy or AraC plus C1498.GM combination therapy. This set of experiments was carried out in Thy 1.1 congenic mice to permit depletion of the tumor cells, which were Thy 1.2⁺. On day 0, Thy 1.1 congenic C57BL/6 mice were intravenously challenged with 2.5×10⁴ live Thy 1.2 C1498-luc leukemia cells. Starting on day 1, three i.p. injections of AraC were administered 10 hours apart to mice designated to receive the combination therapy. On day 7, 1×10⁶ irradiated C1498.GM cells were given as monotherapy alone or to AraC treated mice that received the combination therapy. On day 21, spleen cells from the mice (n=5 per group) were harvested and depleted of the Thy 1.2⁺ C1498-luc tumor cells using anti-Thy1.2 MACs beads. Tumor-depleted splenocytes were confirmed by flow cytometry to contain less than 1% Thy 1.2⁺ C1498-luc cell. Splenocytes were co-cultured with irradiated C1498 cells as stimulators at a 25:1 ratio for five days with five units/mL of murine IL-2 added to the culture on day 2. ⁵¹Cr labeled CTLL2 (a syngenetic lymphoblast cell line) or C1498 cells were used as control and target cells, respectively in the 4 hour ⁵¹Cr release assay. Splenocytes from C1498.GM monotherapy and AraC plus C1498.GM treated mice did not exhibit any cytolytic activity against the CTLL2 control cells at the effector to target ratios evaluated. In contrast, splenocytes from C1498.GM monotherapy or AraC plus C1498.GM treated mice demonstrated comparable cytolytic activity against C1498 target cells which, as expected, was dependent on the effector to target ratio (FIGS. 5B and 5C). Splenocytes from HBSS injected control animals did not demonstrate any cytolytic activity against either of the two target cells. Furthermore, evaluating the tumor-depleted splenocytes for activation markers revealed similar phenotypic patterns for mice treated with C1498.GM monotherapy or AraC plus C1498.GM. Both groups of mice showed a significant increase in the percentage of CD8 cells expressing CD107a⁺ (a marker for CD8 cell cytolytic activity; FIG. 5D), CD44^{hi}CD62L^{1o} (markers for CD8 cell migration and homing; FIG. 5E), and NKG2D⁺ (a CD8 cell activation marker; FIG. 5F) compared to HBSS treated control mice, indicating that, similarly to previous examples of cell-based cancer vaccines employing GM-CSF, the development of the specific immune response was correlated with the activation of CD8 cytotoxic activity. Moreover, the percentage differences in activation markers in the CD8 subpopulation of mice treated with C1498.GM monotherapy compared to AraC plus C1498.GM combination therapy were not significant. Taken together, data from immune monitoring assays suggests that the co-administration of AraC does not interfere with immunotherapy using GM-CSF-secreting cancer cells, and that the combination therapy was successful in inducing specific anti-cancer immune response despite the AraC-induced lymphopeia and neutropenia.

A further indication as to the utility of combining a GM-CSF secreting cellular vaccine and AraC in eliciting an anti-tumor immune response is the production of effector cytokines such as TNF-alpha, IL-2, and IFN-gamma upon restimulation in vitro. Release of such cytokines is often used as a surrogate marker for monitoring tumor specific immune responses following immunotherapeutic strategies designed to induce anti-tumor immunity. Cytokine levels were measured in supernatants from spleen cells restimulated in vitro for 48 hours with irradiated GM-CSF-secreting tumor cells. The presence of the GM-CSF-secreting tumor cells induced the production of TNF-alpha, IFN-gamma, IL-5 and IL-2 by the spleen cells.

What is claimed is:

1. An improved method of cancer therapy, the improvement comprising: administering cytosine arabinoside (AraC) and a cytokine-expressing cancer immunotherapy composition to a subject with cancer, wherein the administration results in enhanced therapeutic efficacy relative to administration of the cytokine-expressing cancer immunotherapy composition or the AraC alone.

2. The method of claim 1, wherein the cytokine-expressing cancer immunotherapy composition comprises cells that express granulocyte-macrophage colony stimulating factor (GM-CSF).

3. The method of claim 2, wherein the cells of said cytokine-expressing cancer immunotherapy composition are autologous to the subject.

4. The method of claim 2, wherein the cells of said cytokine-expressing cancer immunotherapy composition are allogeneic to the subject.

5. The method of claim 2, wherein the cells of said cytokine-expressing cancer immunotherapy composition are bystander cells.

6. The method of claim 2, wherein the cells of said cytokine-expressing cancer immunotherapy composition are rendered proliferation-incompetent by irradiation.

7. The method of claim 2, wherein the subject is a mammal.

8. The method of claim 7, wherein the mammalian subject is a human.

9. The method of claim 2, wherein the cancer is selected from the group consisting of acute myeloid leukemia, prostate cancer, non-small cell lung carcinoma and pancreatic cancer.

10. The method of claim 9, wherein the cancer is an acute myeloid leukemia.

11. The method of claim 4, wherein the allogeneic cells are a cancer-derived cell line, the cell line selected from the group consisting of an acute myeloid leukemia line, a prostate cancer line, a non-small cell lung carcinoma line and a pancreatic cancer line, wherein the cell line is derived from the same type of cancer as the cancer of the subject.

12. The method of claim 11, wherein the allogeneic cells are an acute myeloid leukemia-derived cell line, wherein the cancer of the subject is acute myeloid leukemia.

13. The method of claim 2, wherein the cytokine-expressing cancer immunotherapy composition is administered subcutaneously, intratumorally, or intradermally.

14. The method of claim 13, wherein the cytokine-expressing cancer immunotherapy composition is administered subcutaneously.

15. The method of claim 1, wherein the AraC is administered subcutaneously.

16. The method of claim 1, wherein the AraC is administered intraperitoneally.

17. The method of claim 1, wherein the AraC is administered intravenously.

18. The method of claim 1, wherein the AraC is administered intrathecally.

19. The method of claim 1, wherein administration of the combination results in a long-lasting tumor-specific immune response against the cancer.

20. The method of claim 2, further comprising administration of an additional cancer therapeutic agent or treatment.

21. The method of claim 20, wherein the additional cancer therapeutic agent is expressed by a cell and the cell is an autologous, allogeneic or a bystander cell.

22. The method of claim 21, wherein the autologous, allogeneic or a bystander cell is rendered proliferation-incompetent by irradiation.

23. The method of claim 1, wherein the AraC is administered prior to, at the same time as, or following the administration of the cytokine-expressing cancer immunotherapy composition.

24. The method of claim 23, wherein the AraC is administered prior to the administration of the cytokine-expressing cancer immunotherapy composition.

25. An improved system for cancer therapy, comprising;
a combination of AraC and a cytokine-expressing cancer immunotherapy composition, wherein the combination is co-administered to a subject with cancer, wherein said co-administration results in enhanced therapeutic efficacy relative to administration of the c cytokine-expressing cancer immunotherapy composition or AraC alone.

26. The system of claim 25, wherein the cytokine-expressing cancer immunotherapy composition comprises cells that express granulocyte-macrophage colony stimulating factor (GM-CSF).

27. The system of claim 26, wherein the cells of said cytokine-expressing cancer immunotherapy composition are autologous to the subject.

28. The system of claim 26, wherein the cells of said cytokine-expressing cancer immunotherapy composition are allogeneic to the subject.

29. The system of claim 26, wherein the cells of said cytokine-expressing cancer immunotherapy composition are bystander cells.

30. The system of claim 26, wherein the cells of said cytokine-expressing cancer immunotherapy composition are rendered proliferation-incompetent by irradiation.

31. The system of claim 28, wherein the allogeneic cells are a tumor cell line selected from the group consisting of an acute myeloid leukemia, a prostate tumor line, a non-small cell lung carcinoma line and a pancreatic cancer line, wherein the cell line is derived from the same type of cancer as the cancer to be treated.

32. The system of claim 31, wherein the allogeneic cells are an acute myeloid leukemia-derived cell line, wherein the cancer to be treated is acute myeloid leukemia.

33. The system of claim 25, wherein AraC is administered by the intraperitoneal, subcutaneous, intravenous or intrathecal route.

34. The system of claim 25, wherein the cytokine-expressing cancer immunotherapy composition is administered subcutaneously, intratumorally, or intradermally.

35. The method of claim 25, wherein the AraC is administered prior to, at the same time as, or following the administration of the cytokine-expressing cancer immunotherapy composition.

36. The system of claim 34, wherein the AraC is administered prior to the administration of the cytokine-expressing cancer immunotherapy composition.

37. The system of claim 25, wherein administration of said combination provides a long-lasting tumor-specific immune response against the cancer.
